(19) United States
(12) Patent Application Publication
(10) Pub. No.: US 2026/0235572 A1
(43) Pub. Date: Aug. 13, 2026

(54) LONG TERM PASSIVE VOLATILE ORGANIC COMPOUND SAMPLER SYSTEM

(71) Applicant: The Government of the United States of America, as represented by the Secretary of the Navy, Arlington, VA (US)

(72) Inventors: Thomas J. Boyd, Arlington, VA (US); Richard H. Cuenca, Corvallis, OR (US); Yutaka Hagimoto, Corvallis, OR (US)

(73) Assignee: The Government of the United States of America, as represented by the Secretary of the Navy, Arlington, VA (US)

(21) Appl. No.: 19/532,012

(22) Filed: Feb. 6, 2026

Related U.S. Application Data
(60) Provisional application No. 63/755,780, filed on Feb. 7, 2025.

(51) Int. Cl. G01N 33/18

(52) U.S. Cl. CPC G01N 33/1826

(57) ABSTRACT

Disclosed herein is an apparatus having: a tube, a polydimethylsiloxane plug that divides the tube into an upper portion and a lower portion, and a volatile organic compound trapping device within the upper portion. The apparatus may be used by: positioning an open end of the lower portion into groundwater, equalizing air pressure between the lower portion and the upper portion using a canula, maintaining the open end in the groundwater for a period of time, removing the trapping device, and testing the trapping device for the presence of volatile organic compounds. The concentration of volatile organic compounds in the groundwater may be calculated from the concentration of volatile organic compounds in the trapping device. The apparatus may be reused by replacing the trapping device.

### REFERENCE TO RELATED APPLICATIONS

This application claims the benefit of U.S. Provisional Application No. 63/755,780, filed on Feb. 7, 2025. The provisional application and all other publications and patent documents referred to throughout this nonprovisional application are incorporated herein by reference.

### TECHNICAL FIELD

The present disclosure is generally related to sampling of volatile organic compounds in groundwater.

### DESCRIPTION OF THE RELATED ART

The Navy's and Marine Corps'Environmental Restoration sites are often contaminated with fuels, chlorinated solvents, explosives, and/or heavy metals. Over 3,500 sites are identified within the Navy's responsibility alone. Remedies often require long-term (>30 years) operation and maintenance due to contaminant persistence, site complexity, or a combination of both. Because site managers must balance risk, costs and outcomes that satisfy regulators and stakeholders, there is a persistent need for methodological improvements to provide long-term contaminant fate and transport data. Discreet sampling to obtain contaminant concentrations is expensive and limited in terms of temporal resolution. For instance, quarterly or yearly sampling provides only punctuated information on contaminant transport, dynamics and fate. Passive samplers installed in groundwater wells offer an attractive alternative because they can be deployed for longer time-frames than discreet sampling can provide. They reduce costs by decreasing purge water disposal, equipment use or rental, and time spent on-site by personnel. The disadvantages or limitations of passive in-water samplers are that they must have sufficient sorptive capacity for the analytes in question, may be subject to diffusive filtration, may allow sorbed contaminant biodegradation during longer deployments, may not be able to be deployed in wells with free product and most importantly, are prone to biofouling. Time-averaged sampling is widely needed at contaminated sites, but may be difficult given current sampler limitations.

### SUMMARY

Disclosed herein is an apparatus comprising: a tube, a plug comprising polydimethylsiloxane within the tube that divides the tube into an upper portion and a lower portion, and a volatile organic compound trapping device within the upper portion.

Also disclosed herein is a method comprising: providing the above apparatus, positioning an open end of the lower portion into groundwater, equalizing air pressure between the lower portion and the upper portion using a canula, maintaining the open end in the groundwater for a period of time, removing the trapping device, and testing the trapping device for the presence of volatile organic compounds.

### BRIEF DESCRIPTION OF DRAWINGS

A more complete appreciation will be readily obtained by reference to the following Description of the Example Embodiments and the accompanying drawings.

FIG. 1 shows the sampler configuration.

FIG. 2 shows a photograph of deployment of the sampler into a well headspace.

FIG. 3 shows a photograph of an installed VOC trap.

FIG. 4 shows a logic diagram for how the simulation model(s) are developed and function.

FIG. 5 shows well characteristics used for simulation model(s)

FIG. 6-9 show simulations between groundwater concertation and cVOC collected in the disclosed samplers.

FIG. 10 shows a simulation between groundwater concertation and LNAPLs collected in the samplers.

### DETAILED DESCRIPTION

In the following description, for purposes of explanation and not limitation, specific details are set forth in order to provide a thorough understanding of the present disclosure. However, it will be apparent to one skilled in the art that the present subject matter may be practiced in other embodiments that depart from these specific details. In other instances, detailed descriptions of well-known methods and devices are omitted so as to not obscure the present disclosure with unnecessary detail.

Disclosed herein is an apparatus and method to determine volatile organic compound concentration(s) (contaminants) in groundwater passively over long periods (~3-6 months) without the need to sample the water itself, create hazardous waste and to reduce overall sampling costs.

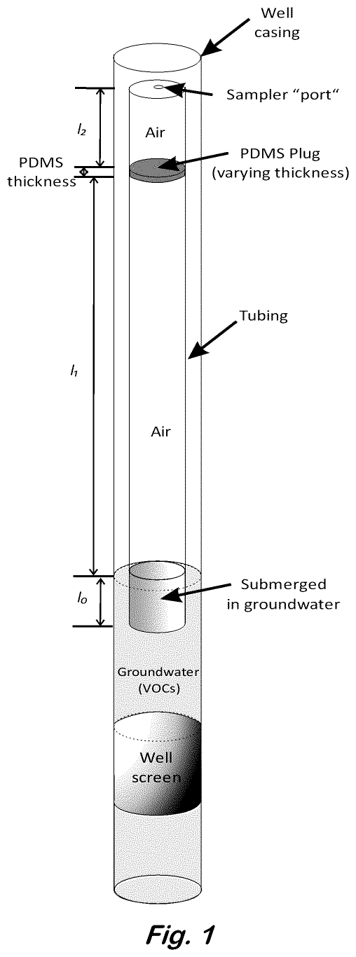

The sampler system may be deployed in a groundwater well casing headspace. Volatile organic compound (VOC) traps fitted to the sampler and residing above the water table eliminate atmospheric interferences and obviate the disadvantages inherent in passive in-water samplers such as biofouling (1), contaminant biodegradation (2), and diffusive effects (3). Furthermore, deployment time limitations can be minimized using a polydimethylsiloxane (PDMS) retarding material within the sampler. In-well diffusive samplers can be deployed to collected VOCs over long durations, such as up to 6 months. By developing a “closed” system deployed in the well headspace, the VOCs must partition into an airspace, travel a known distance, and can be subjected to diffusion-retardant material to arrive at a trap over long periods of time (FIG. 1). The sampler is fitted with a PDMS “plug” having a slower VOC diffusion rate than air or water. The plug length is chosen for 1) the expected VOC concentration and 2) the chosen deployment time. Various dimensions used in a simulation model(s) are determined during deployment l₁ (distance from groundwater to PDMS plug), PDMS plug thickness, l₂ (distance from PDMS plug to sampler) and the depth of groundwater in the sampler (l₀) are determined during deployment (FIG. 1). The sampler inside diameter is also known. Pre-validation estimates of VOC diffusion coefficient(s) and mass transfer velocities in water, air and PDMS (if known) are obtained primarily from the published literature.

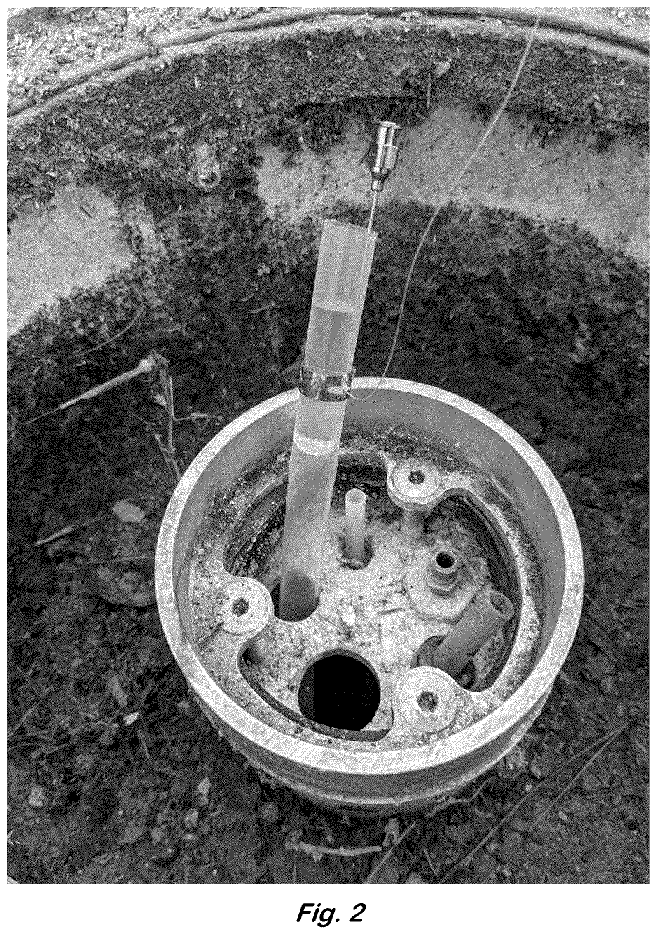

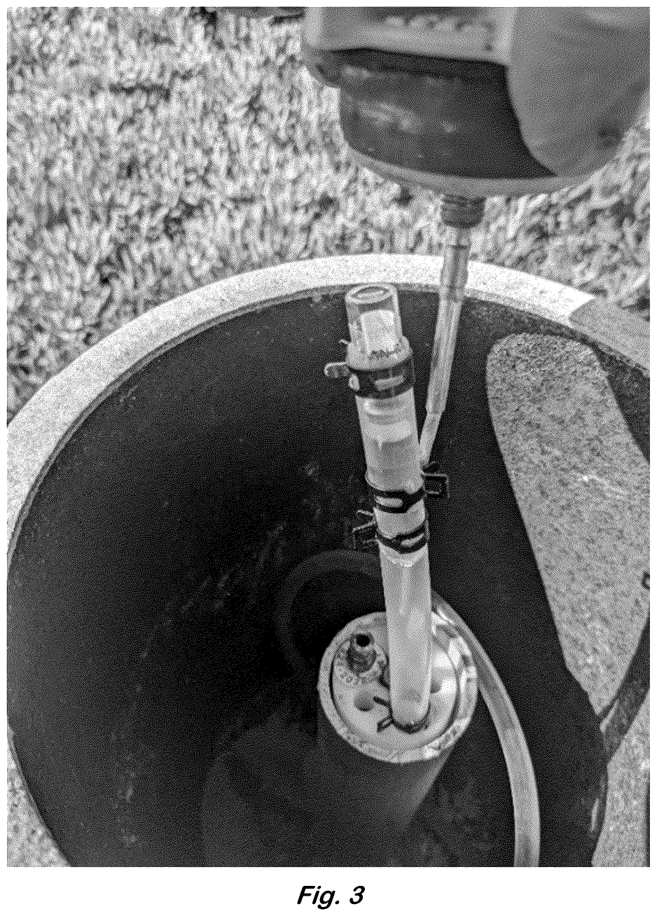

The sampler deployment is straightforward. The tubing may be made of any material that does not allow escape of the VOCs being trapped and measured. The tubing is cut to a specific length based on well depth, depth to water, and screened interval. The tubing is amended with a PDMS plug to retard VOC delivery to the VOC trap. The plug fully divides the tube into an upper and lower portion. The plug should fill the cross-section of the tube, while also allowing for the insertion of a canula alongside the plug to equilibrate head pressure. The tube is then lowered to the desired depth within the well casing (FIG. 2). A tether as necessary is installed and the PDMS plug is secured with a tubing clamp (FIG. 2). The VOC trap is then installed into the tube and secured (FIG. 3). Such traps are known in the art and are available commercially. The PDMS plug inhibits the transfer of water vapor to the VOC trap and significantly slows the diffusion of VOCs into the trap (increasing the possible deployment time). After the desired deployment time, the VOC traps are removed and sent for chemical analysis. For long-term monitoring, a fresh VOC trap can be added and the well resealed for recovery at a later date.

The device was developed and tested using a commercially-available VOC trap device (SiREM Waterloo Membrane Sampler™ (WMS™)), which has been validated for soil gas sampling and analysis. These VOC traps are fixed cost, readily available and can be ordered with full chemical analysis. Once removed, these VOC traps are sealed (with desiccant) and mailed to the manufacturer for chemical analysis.

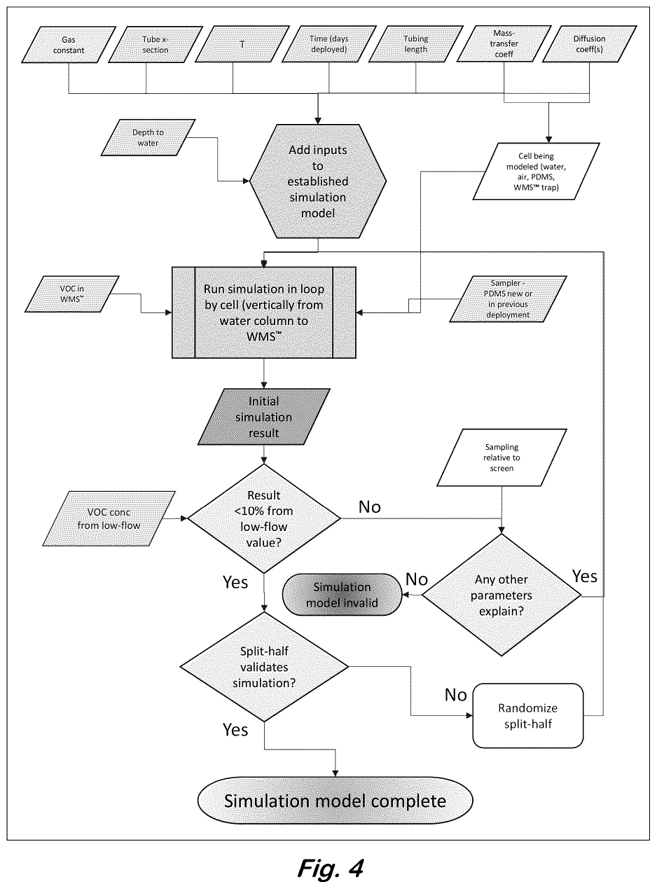

The dimensions of the sampler may be determined using a simulation model. FIG. 4 shows a logic diagram for how the simulation model(s) are developed and function. Simulation models take in various constant data, either derived from the literature or empirically during sampler prototyping in the laboratory. Data constants include the universal gas constant (R), mass transfer coefficient(s) for the various VOC measured, and apparent or effective diffusion coefficient(s) for the VOC measured. Field or variable data include depth to water, tube cross section (area), temperature, tubing length, and VOCs measured in the WMS™ VOC trap. Additionally, if the sampler and PDMS were installed during a previous sampling, the headspace and PDMS will be at equilibrium with VOCs in the tube headspace and the cells representing these areas within the simulation model will run as such. Otherwise, mass transfer and diffusion coefficients will drive diffusion rate estimates for VOCs through those cells to the WMS™ VOC trap interface (which is a trap driving equilibrium). These inputs drive the simulation model and an initial result is derived (FIG. 4).

The simulation output should optimally be the same (within 10%) as the low-flow VOC concentration estimate (from previous long-term sampling). If not (see first decision point in FIG. 4), subjective information like the tendency to over or underestimate based on the sampler tube end relative to the screen and low-flow sampling depth may be input to the simulation model to drive it toward convergence. The full dataset is first divided into calibration and validation subsamples of equal size, defined by sequential sample number to ensure fairness. Convergence and model calibration are performed using only the calibration subsample. The calibrated model is then evaluated using the validation subsample to assess model performance. If the validation results are not within acceptable limits (2nd decision point in FIG. 4), subjective factors may again be applied to the simulation model, and convergence and model calibration are repeated using the same calibration subsample. The updated model is then re-evaluated using the same validation subsample. If this process provides convergence and acceptable validation performance, the simulation model is considered validated and may be used for subsequent relation of NESDI sampler results to low-flow concentration estimates. If the simulation model continues to fail convergence or validation (<10%), it will be considered invalid, unless RPMs, regulators, or stakeholders determine it to be sufficient for the intended purpose (FIG. 4).

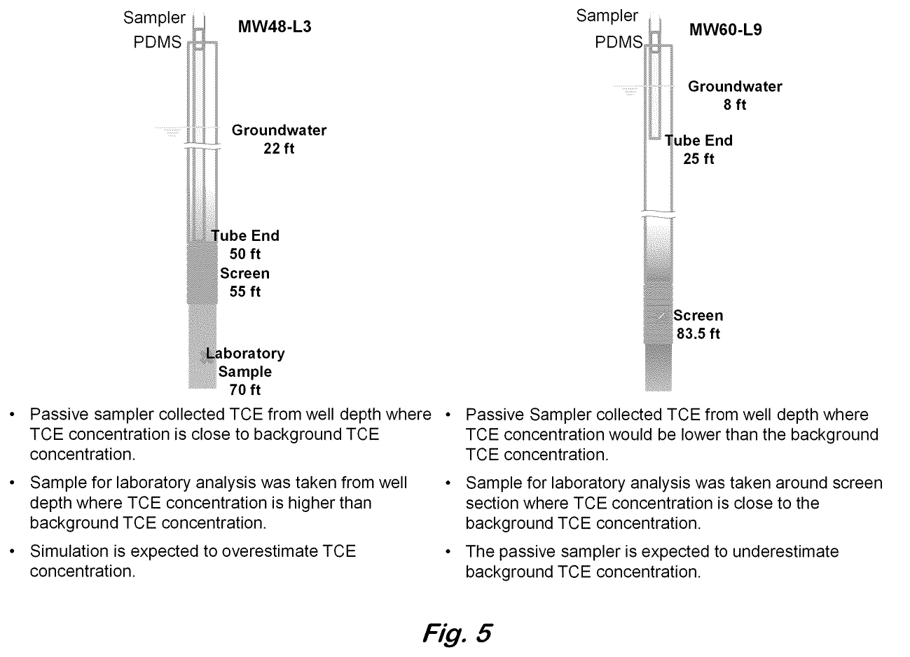

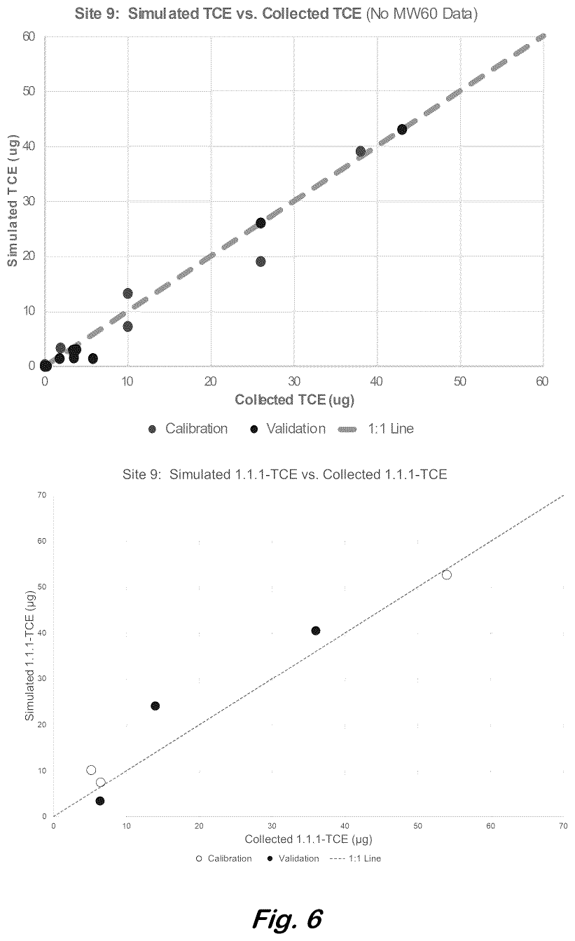

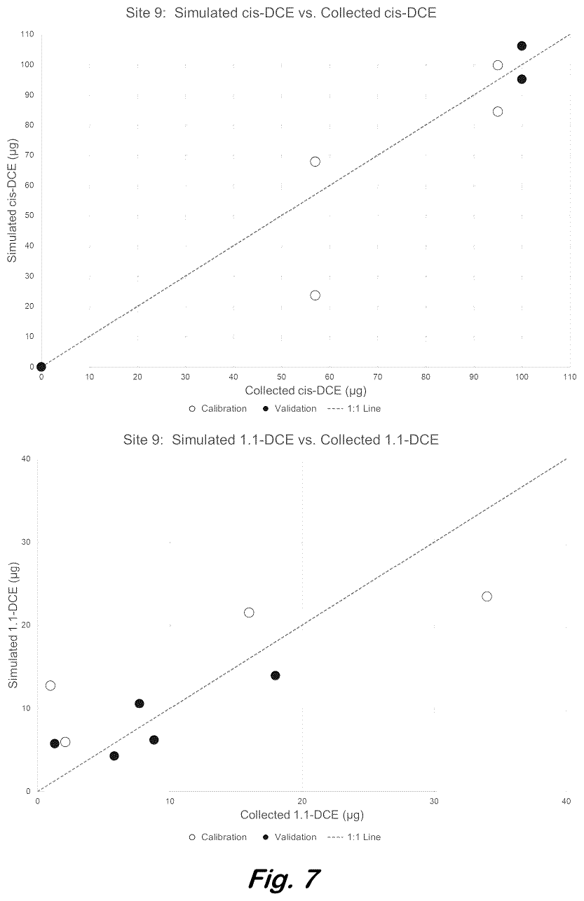

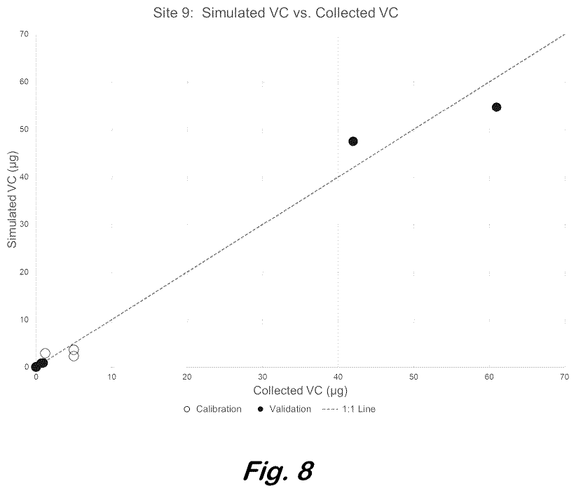

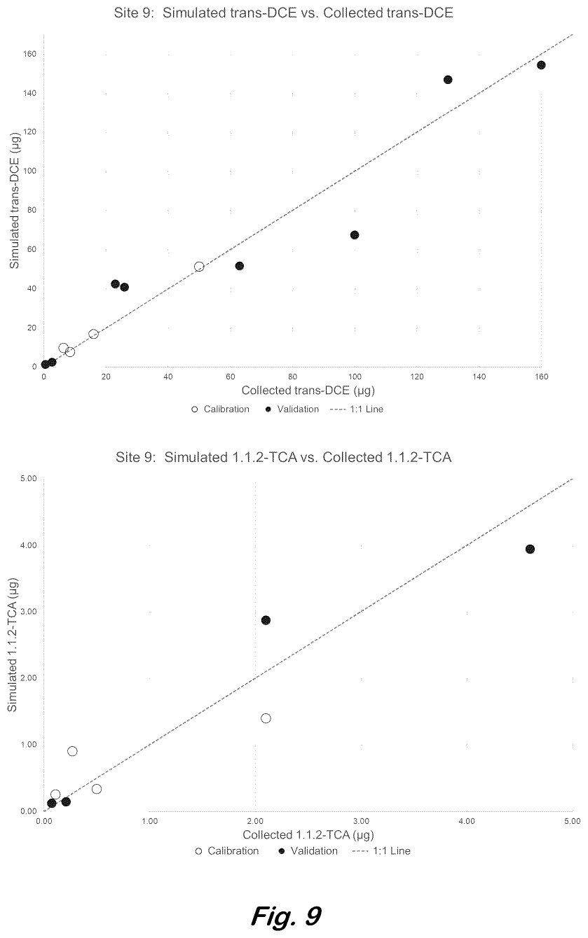

A simulation model was developed and is used to convert groundwater VOC centration to observed results based on sampler geometry, deployment depth, PDMS plug length, deployment time, and relevant chemical and physical data [temperature, VOC diffusion coefficient(s) and mass transfer coefficients]. The simulation relies on Henry's Law to calculate VOC mass transfer dynamics from groundwater, into the sampler air column, through PDMS and into the WMS™ VOC trap. The simulation model also accounts for any differences between the low-flow sampling geometry and the tube ending of the sampler (FIG. 5). Test deployments in wells with no associated free product (pools of contaminant not dissolved in groundwater) over 4 three-month periods were able to be modeled with a significant (P>0.05) correlation offering a 0.99 goodness of fit statistic (FIG. 6). Wells with very high VOC concentrations (indicating free product) were modeled less effectively (R²=0.62). Split-half validation was used. Multiple chlorinated volatile organic compounds (cVOCs) were measured both in groundwater (low-flow) and the traps over time at Site 9 and for one year at Site 2 (Saint Juliens Creek Annex in Portsmouth, VA). 1,1,1-TCE, cis-DCE, 1,1-DCE and VC were simulated at Site 9 (FIGS. 6-9). Results (goodness of fit statistic: r²) for the simulation relative to low-flow cVOC results were above 0.8 for TCE (0.99), 1,1,1-TCE (0.92), cis-DCE (0.89) and VC (0.98). Results for 1,1-DCE were more problematic with a validation fit of 0.62. On-site concentration values for this contaminant were lower relative to other cVOCs sampled over the 2-year deployment and sampling period. At Site 2 (SJCA), Simulations have been developed for 1,1,2-TCA and trans-DCE (FIG. 9). Goodness of fit between low-flow data and the samplers are 0.89 for 1,1,2-TCA and 0.93 for trans-DCE. These are above the ~25% analytical uncertainty typical for low-flow cVOC analysis. As a reminder, the simulations for all components other than TCE have been completed with estimated diffusion coefficients. TCE diffusion was modeled in the test samplers in the laboratory before deploying in the field. Results are within the typical uncertainty for field low-flow VOC sampling and analysis.

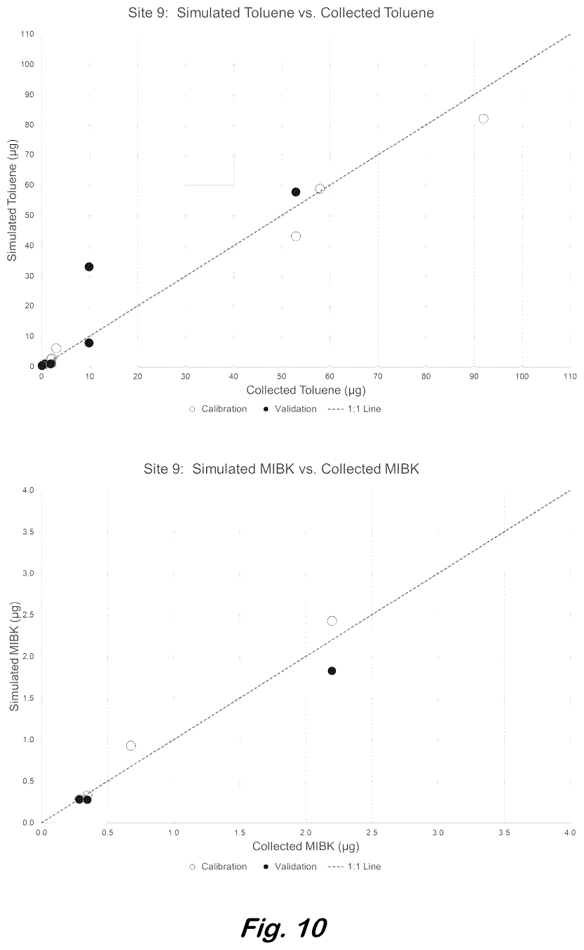

In addition to cVOCs, the sampler was evaluated for non-chlorinated volatile compounds. The cVOCs evaluated were all dense non-aqueous phase liquid (DNAPL) chemicals—meaning they sink in water and form a phase layer in groundwater above a non-porous layer (aquitard). Most non-chlorinated organic groundwater contaminants are light non-aqueous phase liquids (LNAPLs) and float at the groundwater surface. Fuels are notable LNAPLs; this is the basis for fuel-water separators having a drain at the bottom of the bowl—where any denser water in fuel will accumulate. LNAPL compounds thus differ in how they interact with the sampler relative to well screening and the tube depth. LNAPL components also have relatively low Henry's law constants. Simulations were altered accordingly. Two LNAPL components were simultaneously measured in both groundwater via low-flow sampling and by the sampler at Site 9, toluene and 4-methyl-2-pentanone (MIBK). Simulations were developed for these components and split-half validation yielded goodness of fit above 0.94 for both compounds (FIG. 10). The sampler thus has universal applicability for all volatile organic contaminants commonly found in groundwater.

Potential advantages of the sampler are 1) enhanced flexibility in compliance monitoring for VOCs in groundwater (able to be deployed for varying time periods); 2) avoidance of the main drawbacks to in-water passive samplers (biofouling (obviated using sealed VOC soil gas samplers, contaminant biodegradation (no exposure of sorbed contaminants to the VOC soil gas samplers and no microbial growth on surfaces that are not immersed in groundwater, and diffusive effects (sampler column consisting of air, water and PDMS are contained within the tubing column without any lateral diffusion; 3) ease of deployment which takes less than 15 minutes at “new” wells (e.g. measuring and adding tubing to well headspace) and less than 5 minutes at wells with sampler infrastructure installed (simply swap the VOC soil gas sampler); 4) lower overall cost (10 wells sampled by traditional low-flow techniques costs ~$50K (per NavFac site managers). The sampler costs less than $1,000); 5) no generation of hazardous waste (purge water for low-flow sampling) which minimizes overall environmental impact. It relies on a vetted air sampler for VOCs (SiREM Waterloo Membrane Sampler™ (WMS™)) which has DoD certification for soil gas monitoring; and 6) compliance with current ASTM methods for passive sampling (ASTM D7929-20).

Features of the sampler include that the VOC monitoring media never comes in contact with the monitored groundwater—thus not being exposed to microbes, scaling or other in-water limitations to passive sampling. It relies on a soil gas VOC trap to monitor groundwater VOC concentrations and is useful because it can reduce costs for groundwater VOC monitor and can be used for long periods relative to other samplers currently available and can be modified for monitoring multiple VOCs.

Current alternatives include traditional low-flow sampling techniques which are costly, time-consuming and generate significant hazardous waste. Other passive sampler use has been hindered by biofouling, poor validation results, diffusive effects or limited deployment timeframes. No passive samplers described in the literature are able to be reliably deployed for 3-6 month timeframes to integrate VOC groundwater concentrations.

Many modifications and variations are possible in light of the above teachings. It is therefore to be understood that the claimed subject matter may be practiced otherwise than as specifically described. Any reference to claim elements in the singular, e.g., using the articles “a”, “an”, “the”, or “said” is not construed as limiting the element to the singular.

### ABBREVIATIONS

TCE Trichloroethylene
1,1-DCE 1,1-Dichloroethylene
cis-1,2-DCE cis-1,2-Dichloroethylene
trans-1,2-DCE trans-1,2-Dichloroethylene
1,1,1-TCA 1,1,1-Trichloroethane
1,1,2-TCA 1,1,2-Trichloroethane
VC Vinyl Chloride

What is claimed is:

1. An apparatus comprising:
a tube;
a plug comprising polydimethylsiloxane within the tube;
wherein the plug fully divides the tube into an upper portion and a lower portion; and
a volatile organic compound trapping device within the upper portion.

2. The apparatus of claim 1, further comprising:
a canula inserted between the plug and the tube to equalize air pressure between the upper portion and the lower portion.

3. A method comprising:
providing the apparatus of claim 2;
positioning an open end of the lower portion into groundwater;
equalizing air pressure between the lower portion and the upper portion using the canula;
maintaining the open end in the groundwater for a period of time;
removing the trapping device; and
testing the trapping device for the presence of volatile organic compounds.

4. The method of claim 3, further comprising:
calculating a concentration of volatile organic compounds in the groundwater from a concentration of volatile organic compounds in the trapping device.

5. The method of claim 3, further comprising:
placing a second volatile organic compound trapping device into the upper portion;
maintaining the open end in the groundwater for a second period of time;
removing the second trapping device; and
testing the second trapping device for the presence of volatile organic compounds.

6. The method of claim 5, further comprising:
calculating a second concentration of volatile organic compounds in the groundwater from a concentration of volatile organic compounds in the second trapping device.
